(19) United States
(12) Patent Application Publication
(10) Pub. No.: US 2010/0057173 A1
(43) Pub. Date: Mar. 4, 2010

(54) Versatile System for Therapeutic Thermal Reduction

(76) Inventor: William A. Leavitt, Frisco, TX (US)

Correspondence Address: PATTON BOGGS, LLP, 2001 ROSS AVENUE, SUITE 3000, DALLAS, TX, 75201, US

(21) Appl. No.: 12/202,575

(22) Filed: Sep. 2, 2008

(51) Int. Cl. A61F 7/00

(52) U.S. Cl. 607/114

(57) ABSTRACT

A system provides therapeutic reduction of, and relief from, discomfort and inflammation stemming from strenuous physical activity. The system provides a therapeutic icing apparatus that placed in position along a desired area of an individual's body. The apparatus has a receptacle component to hold an icing medium, and compress that icing medium against the desired area. A positioning component is included to maintain the receptacle component in a desired orientation, and to optimize the individual's comfort and mobility. One or more compression components are provided to optimize compression of the receptacle component, without causing discomfort to the individual. The materials and configuration of the system are provided to optimize the adaptability, effectiveness and convenience of the system.

### TECHNICAL FIELD OF THE INVENTION

This invention relates, in general, to therapy systems and, in particular, to a versatile system for providing optimal therapeutic cooling over an extensive area of a body in a convenient and highly efficient manner.

### BACKGROUND OF THE INVENTION

There are a number of competitive, sports and athletic activities that require forceful, repetitive motion. Such activities may include, for example: pitching a baseball; throwing a football; running a race; golfing; serving a tennis ball; and swimming. As a result, participants in such activities—at all levels, from amateur to professional—can experience varying degrees of physiological stress or inflammation that may affect or inhibit their performance and participation.

For example, during the course of a baseball game, a pitcher may strenuously throw a baseball 50 times or more. Such repetitive strain causes soreness and inflammation in the shoulder and arm of a player—as lactic acid builds up in the muscle tissue, and joints, tendons and muscles all become irritated and inflamed. As a result, the player may experience anything from mild discomfort that degrades on-field performance, to debilitating pain that precludes further activity.

It is commonly understood that intense cooling (or “icing”) of an inflamed or sore body part can provide therapeutic benefits by reducing and relieving discomfort and inflammation. Parents, coaches, trainers and players have conventionally relied upon various icing techniques to provide such relief from sports-induced pain and discomfort, and to reduce adverse effects of sports-induced stress and strain. Typically, this took the form of placing a cold compress or an ice-bag (collectively, “compress”) on an area of the body that was sore, or prone to becoming sore. This approach was somewhat inconvenient—because someone, be it the player or another person, was usually required to hold the compress on an affected area. This approach was also limited in its scope, because the area of the body being “iced” was only as big as the compress itself. If a larger area of the body was to be treated, an individual either had to try to hold multiple compresses simultaneously, or move a single compress around periodically.

Attempts were made to address these issues. Most such attempts took the general form of one of the two approaches illustrated with reference now to FIGS. 1(a) and 1(b). FIGS. 1(a) and 1(b) generally depict the torso 100 of a person having a conventional icing assembly disposed to provide relief to the shoulder/upper-arm area 102. Referring to FIG. 1(a), the conventional icing assembly takes the form of a bag of ice 104 disposed somewhere within area 102 and held in position by some form of wrap 106. Ice bag 104 may be a simple as a plastic grocery or zip-seal bag, or it may actually be a conventional ice bag apparatus designed specifically to hold a limited amount of ice for medical purposes (e.g., headache relief). Some limited quantity of ice is placed into bag 104, which is then positioned somewhere in area 102 to provide therapeutic benefit.

Bag 104 may be held in place manually by an individual; or some type of wrap 106 may be utilized to position or hold bag 104 in a desired location or orientation. Wrap 106 usually takes the form of an elastic bandage, gauze, or some type of plastic wrap that is stretched repetitively around torso 100 and bag 104.

Typically, this arrangement provides a great deal of flexibility when considering the positioning of bag 104. Bag 104 may be placed just about anywhere on the body, in a wide range of orientations, so long as wrap 106 can be positioned to hold it in place sufficiently. Furthermore, the fact that this approach utilizes actual ice provides a potentially high degree of conformity to the contours of area 102. Depending upon the granularity of the ice used, bag 104 may be positioned and manipulated to establish direct contact with a wide variety of shapes and features of area 102, providing a high level of therapeutic benefit. Also, ice is—generally speaking—conveniently ready in a variety of forms in many locations (e.g., snack bars, convenience stores), and can be quickly and easily replenished in a relatively short amount of time.

Even so, there are a number of ways in which this approach is not optimally convenient or effective. There can be a great degree of thermal loss through the ice bag and wrap, depending upon the materials utilized—reducing the effectiveness of the icing process. Utilizing this approach to ice an extensive area can prove to be cumbersome, at best, and impossible, at worst. Concurrent icing of extensive areas of a body (e.g., the combined pectoral, shoulder and scapular areas on one side of the body) requires a large ice bag surface area. Placement of multiple standard ice bags—or even a single large ice bag (e.g., a trash bag)—and securing those bags in place with a wrap can be a laborious and time-consuming process. As such, icing by this approach may not be utilized during rest periods in the course of an event, and may be employed only after an event has concluded. Even where such awkward placement and wrapping is accomplished, full coverage of a desired area still may not be achieved. Furthermore, for the individual receiving the icing, this approach can be uncomfortable and inconvenient. Typically, once an individual has been wrapped for icing in this manner, they need to remain relatively sedentary or stationary because the ice bag and its wrapping are likely to shift or slide due to the effects of gravity, and especially if the individual moves around. Wrapping can pinch, bind and chafe the individual, and may restrict the movement of body parts not being iced.

Another conventional icing assembly is described now in reference to FIG. 1(b). This conventional assembly attempts to overcome some of the deficiencies of approach illustrated in FIG. 1(a), but then causes other problems of its own. In this approach, the icing assembly takes the form of a compress 108 disposed somewhere within area 102, and held in position by one or more straps 110. Compress 108 typically comprises some sort of re-freezable medium—such as a gel pack or water bag. The re-freezable medium is separated or removed from the assembly, and kept in a freezer or refrigeration unit until needed. When icing is commenced, the medium is removed from refrigeration and reattached to straps 110, or placed in some sort of pouch to which straps 110 are already attached. The assembly is then positioned in a desired location within area 102, and secured in place with straps 110.

This approach usually overcomes the comfort, and certain convenience, limitations of the previously described approach. Individuals typically experience lesser, or no, restriction on their movement while icing is in progress. The straps generally keep the compress well-positioned even if the individual is active and moving. Also, in most cases, the straps tend not to bind or pinch to the same extent that a wrap 104 does. Even so, this approach does have a number of disadvantages.

For example, most compresses 108 tend to be small to moderate in size, and thus effective for icing only a limited, localized area. Because the re-freezable medium has to be of a size that can easily fit within a freezer or refrigeration unit, compress 108 is typically not of a sufficient size to concurrently ice an extensive area of the body. Furthermore, once the re-freezable medium is removed from refrigeration, it is generally rigid in nature and does not readily conform to contours and shapes of icing area 102. This results in a reduced therapeutic effect for the icing process. Thermal loss due to the materials used for the compress can further degrade the therapeutic effects of this approach.

Once a compress 108 has been used for any length of time, it typically requires re-freezing before it can be reused. This renders it inconvenient for repetitive or prolonged use by multiple individuals. As a result, multiple re-freezable mediums must be on location if this icing approach is to be used on-site during an event. Further inconvenience then stems from the transport of extra material (i.e., multiple re-freezable mediums) and the need to have access to on-site refrigeration.

As such, there arises a need for a versatile system that provides optimal therapeutic cooling, over an extensive area of the body, in a convenient and highly efficient manner.

### SUMMARY OF THE INVENTION

The present invention disclosed herein provides a versatile system for providing optimal therapeutic cooling over an extensive area of the body, in a convenient and highly efficient manner. The present invention recognizes that optimal therapeutic cooling (or icing) of a part or area of a body depends on several factors, and addresses each to maximize icing effectiveness. At the same time, the present invention achieves these ends with a system that is comfortable, convenient and easy to use.

The system of the present invention provides numerous structures and methods, including a therapeutic icing apparatus that may be utilized to treat an extensive area of individual's body. The present invention provides a receptacle component to hold an icing medium—whether that be ice, re-freezable mediums (e.g., gels), or removable pouch or bladder—and compress that icing medium against the desired area. A positioning component maintains the receptacle component in a desired orientation, and optimizes the individual's comfort and mobility. One or more compression components are provided to optimize compression of the receptacle component, without causing discomfort to the individual. The materials and configuration of the system are provided to optimize the adaptability, effectiveness and convenience of the system.

In one aspect, the present invention provides a system for therapeutically cooling a desired area on an individual's body. The system comprises a therapeutic medium receptacle, having insulating and compression features. A positioning component is connected to the therapeutic medium receptacle, and adapted to maintain the therapeutic medium receptacle in a desired orientation with respect to the desired area. A compression component is connected to the therapeutic medium receptacle, and adapted to facilitate compression of the therapeutic medium receptacle against the desired area.

In certain embodiments, a therapeutic medium may be loaded directly into therapeutic medium receptacle. In other embodiments, a container component is provided and a therapeutic medium is loaded therein. That container component is provided in a manner such that it may be easily disposed within the therapeutic medium receptacle.

In another aspect, the present invention provides a method of therapeutically cooling a desired area on an individual's body. A therapeutic medium receptacle is provided with insulating and compression features. A positioning component is connected to the therapeutic medium receptacle, and adapted to maintain the therapeutic medium receptacle in a desired orientation with respect to the desired area. A compression component is connected to the therapeutic medium receptacle, and adapted to facilitate compression of the therapeutic medium receptacle against the desired area. Therapeutic medium is loaded into the therapeutic medium receptacle, either directly or within a container component, and the therapeutic medium receptacle, the positioning component, and the compression component are secured in place on the individual's body.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIGS. 1(a) and 1(b) are diagrams illustrating prior art materials and methods for therapeutic icing;

FIG. 2 is a diagram illustrating one embodiment of a therapeutic icing system according to certain aspects of the present invention;

FIG. 3 is a diagram illustrating additional aspects of the embodiment illustrated in FIG. 2;

FIG. 4 is a diagram illustrating one embodiment of a receptacle component according to certain aspects of the present invention;

FIG. 5 is a diagram illustrating a cross-sectional view of one embodiment of a receptacle panel according to certain aspects of the present invention;

FIG. 6 is a diagram illustrating one embodiment of a position component according to certain aspects of the present invention;

FIG. 7 is a diagram illustrating one embodiment of a therapeutic icing system according to certain aspects of the present invention;

FIG. 8 is a diagram illustrating one embodiment of a container component according to certain aspects of the present invention;

FIG. 9 is a diagram illustrating another view of the container component depicted in FIG. 8;

FIGS. 10(a)-10(c) are diagrams illustrating one embodiment of a loading assembly according to certain aspects of the present invention; and

FIGS. 11(a) and 11(b) are diagrams illustrating another embodiment of a therapeutic icing system according to certain aspects of the present invention.

### DETAILED DESCRIPTION OF THE INVENTION

While the making and using of various embodiments of the present invention are discussed in detail below, it should be appreciated that the present invention provides many applicable inventive concepts which can be embodied in a wide variety of specific contexts. For example, certain embodiments of the present invention are illustrated and described in relation to therapeutic cooling of the shoulder region of a human torso. The principles and practices of the present invention may also be applied to other areas or parts of the body—such as the knee region or lower back area, for example. The principles and practices of the present invention may also be applied to non-human uses, such as race horse therapy. Furthermore, certain embodiments of the present invention are described and illustrated in reference to specific materials, even though numerous other suitable materials may also be utilized in accordance with the present invention. Therefore, the specific embodiments discussed herein are merely illustrative of specific ways to make and use the invention, and do not delimit the scope of the invention.

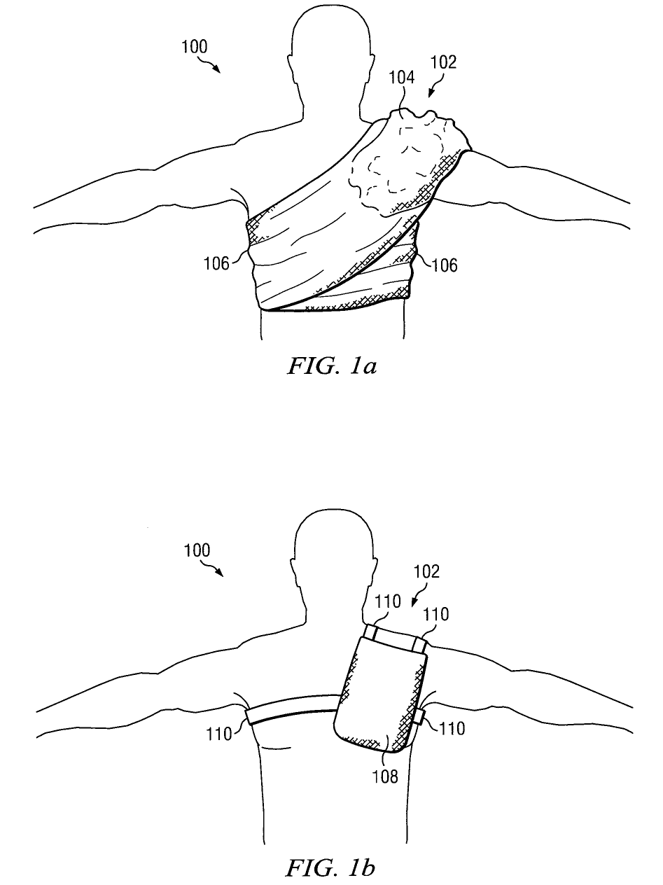

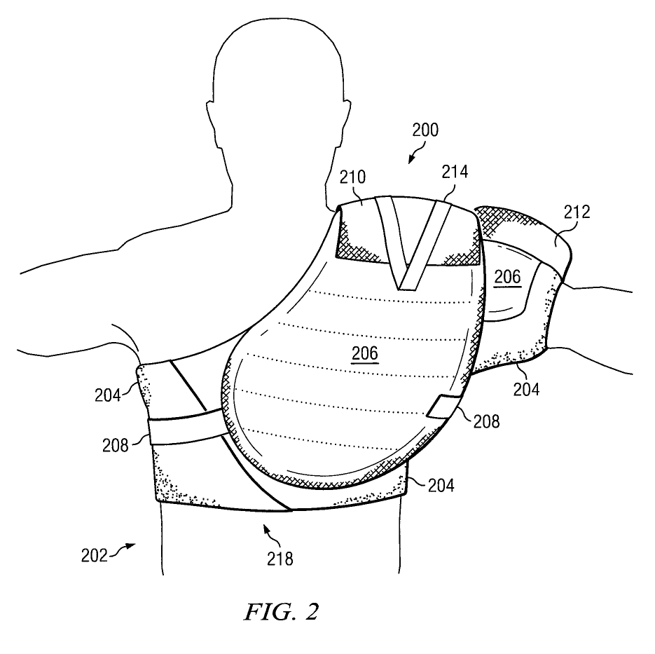

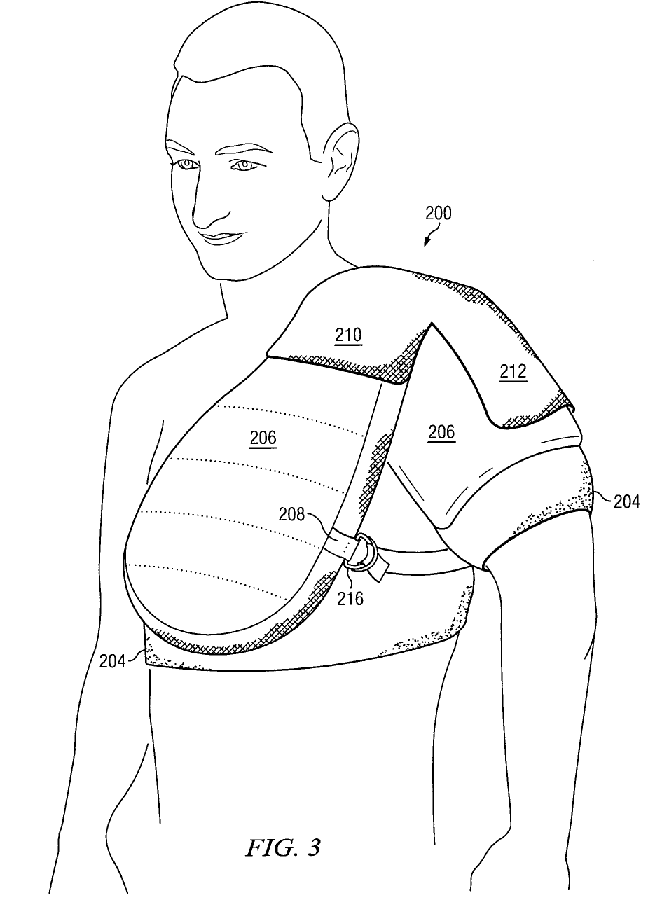

Referring initially to FIGS. 2 and 3, one embodiment of a therapeutic cooling apparatus 200 according to the present invention is illustrated. Apparatus 200 is illustrated in reference to therapeutic cooling (hereinafter “icing”) of the shoulder region of torso 202, so as to provide illustrative comparison to the prior art approaches previously illustrated in FIGS. 1(a) and 1(b). It should be clearly understood, however, that other configurations in accordance with the following specification may be provided for icing of other body parts or regions.

Apparatus 200 comprises: one or more positioning component(s) 204; one or more medium receptacle(s) 206 secured to, or integrated with, positioning component(s) 204; and one or more compression component(s) 208 secured to, or integrated with, medium receptacle(s) 206 and aligned with positioning component(s) 204.

Medium receptacle(s) 206 are formed and fabricated to receive and secure an icing medium (e.g., ice)—either directly, or within a container component—and to place the icing medium in close or direct contact with a desired area of the body part 202. Medium receptacle(s) 206 are formed and fabricated to provide compressive force sufficient to maintain the icing medium in therapeutic contact with the desired area. Medium receptacle(s) 206 are configured as a pouch, sheath, pocket, shell or other suitable receptacle for receiving an icing medium. In some embodiments, an icing medium may be deposited directly into receptacle 206. In other embodiments, receptacle 206 may be configured to receive a container component—such as a bladder, bag or other similar component—that contains the icing medium. In embodiments where an icing medium is deposited directly into receptacle 206, the interior surfaces of the receptacle may be comprised of or covered with a suitable waterproof material, so as to prevent messes or damage from melting icing medium. For purposes of explanation and illustration, however, the description hereafter references embodiments where receptacle 206 is configured to receive a component that contains the icing medium (referred to hereinafter as “container component”).

In the embodiment depicted in FIG. 2, receptacles 206 are formed of sufficient size and shape so as to provide therapeutic icing coverage to the entire pectoral, upper shoulder and scapular area of torso 202. Although receptacles 206 are formed to provide such extensive treatment, they may also be selectively used to treat a smaller area. In the illustrated embodiment, one large symmetrical receptacle 206 is formed in a “saddlebag” shape so as to extend from front to back evenly. This receptacle 206, and this embodiment of apparatus 200, is operationally symmetrical such that a single apparatus may be worn over either the left or right shoulder of an individual. Another smaller, symmetrical receptacle 206 is attached to or integrated with the larger receptacle, so as to extend coverage of the apparatus over the deltoid region of torso 202.

Apparatus 200 further comprises coverings 210 and 212 that are movably disposed over the open portions of receptacles 206. Covering 210 comprises a large flap attached to or integrated with the large receptacles 206 in such a manner that it extends over the crest of the shoulder area of apparatus 200. Covering 210 encloses an icing bladder within large receptacle 206—securing the icing bladder in place, and providing thermal and compression benefits similar to those of receptacle 206. Covering 206 is illustrated hereinafter in greater detail. Apparatus 200 may comprise some form of attachment or closure member 214 that keeps covering 210 closed securely during use of the apparatus. Covering 212 comprises a smaller flap that extends from covering 210 outwardly over smaller receptacle 206—enclosing the icing bladder therein, and providing the same benefits as covering 210. In alternative embodiments, covering 212 may be affixed to receptacle 206, or removably attached to covering 210.

Apparatus 200 comprises one or more positioning components 204 that may be contiguously formed, or individually formed and integrated or attached. Positioning components 204 are integrated with, or attached to, receptacle(s) 206. They are formed of size, shape and material to maintain or hold receptacle(s) 206 in a desired orientation with respect to torso 202. The formation and material selection for positioning component(s) 204 may also factor in several convenience or comfort factors—such as optimizing comfort of an individual wearing apparatus 200, or maximizing mobility the individual while wearing apparatus 200. In the embodiment depicted, positioning component(s) 204 are formed of neoprene—or some similar substance such as Lycra™, rip-stop nylon, etc.—that provides sufficient firmness or rigidity to maintain the positioning of receptacle(s) 206 while providing some degree of comfort to the individual. In this particular embodiment, positioning component(s) 204 provide a sleeve and harness-type foundation for apparatus 200. The closure and securing of positioning component(s) 204 around the torso is described in greater detail hereinafter.

Compression components 208 are attached to, and work in conjunction with, receptacle(s) 206 to maximize contact and contouring of the icing medium along the desired area of torso 202. Compression components 208 comprise straps that may be permanently or removably attached to receptacle(s) 206. In the embodiment depicted, compression components 208 comprise nylon straps that are affixed to the front and back portions of large receptacle 206, and secure together at the sides of torso 202 with friction-type buckles 216. As such, the amount of tension provided by components 208 may be easily adjusted. Other embodiments of the present invention may comprise other connectors (e.g., snap-lock buckles, Velcro™) and other strapping materials (e.g., canvas straps, elastic straps, elastic cords).

Tension provided by compression components 208 works in conjunction with certain features of receptacle(s) 206, described hereinafter, to optimize the inward pressure applied to an icing medium disposed with the receptacle(s). This pressure provides optimal contouring of the icing medium to the body part being iced, and provides optimal icing contact across the area being treated. In some embodiments, compression components may be provided in conjunction with other receptacles—such as around the small shoulder receptacle, for example.

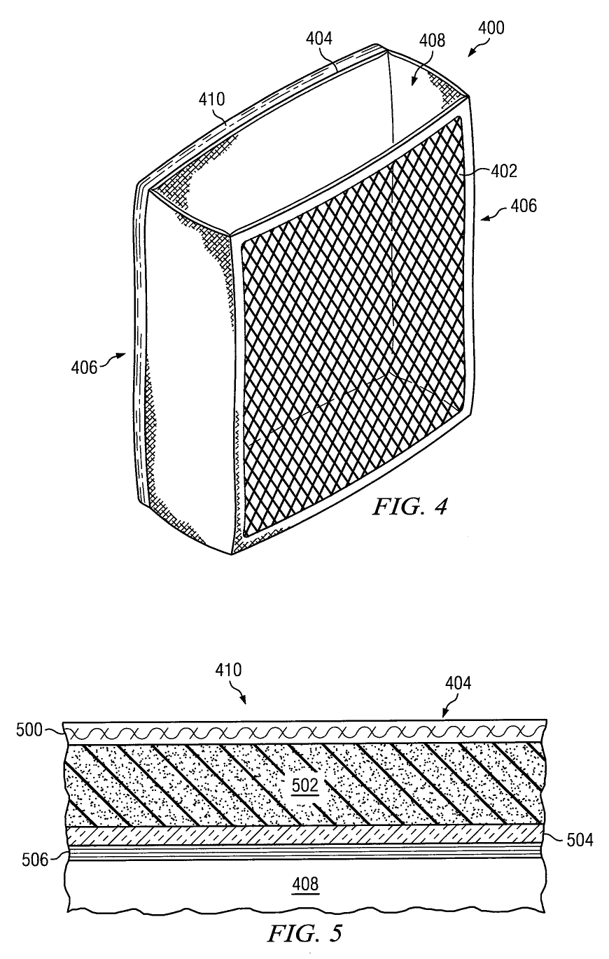

A receptacle according to the present invention is explained with reference now to FIG. 4, which depicts an illustrative, but generalized, embodiment of a receptacle 400 in accordance with the present invention. Receptacle 400 comprises an inward face 402 and an outward face 404, as well as an exterior area 406 and an interior area 408. When in use in an apparatus of the present invention, inward face 402 is the portion of the receptacle that is in direct contact with (or in closest proximity to) an area of a body under treatment. Outward face 404 is the outermost surface of the exterior portion of the receptacle. Interior area 408 is that area inside the pouch, pocket or sheath formed by receptacle 400, within which an icing medium or icing bladder is contained. Exterior area 406 is that area around the outer perimeter of receptacle 400, along which inward face 402 and outward face 404 are disposed. In some embodiments, inward face 402 may comprise a mesh, thin nylon, or other suitable material that is sturdy or secure enough to retain an icing medium or icing bladder, and thermally penetrable to provide optimal thermal exposure of the icing medium or icing bladder to the area of the body under treatment.

Receptacle 400 further comprises an exterior panel 410 disposed along outward face 404, between outward face 404 and interior area 408. Panel 410 comprises a plurality of layers that provide thermal insulation, to minimize melting of an icing medium, as well as a rigid or inflexible quality to enable application of compressive force to interior area 408. When compressive force is applied to the outward face 404 by compressive components, force is transferred via panel 410 to the icing medium within interior area 408—resulting in optimal contouring of the icing medium to the body part being iced, and optimal icing contact with the area being treated, via inward face 402.

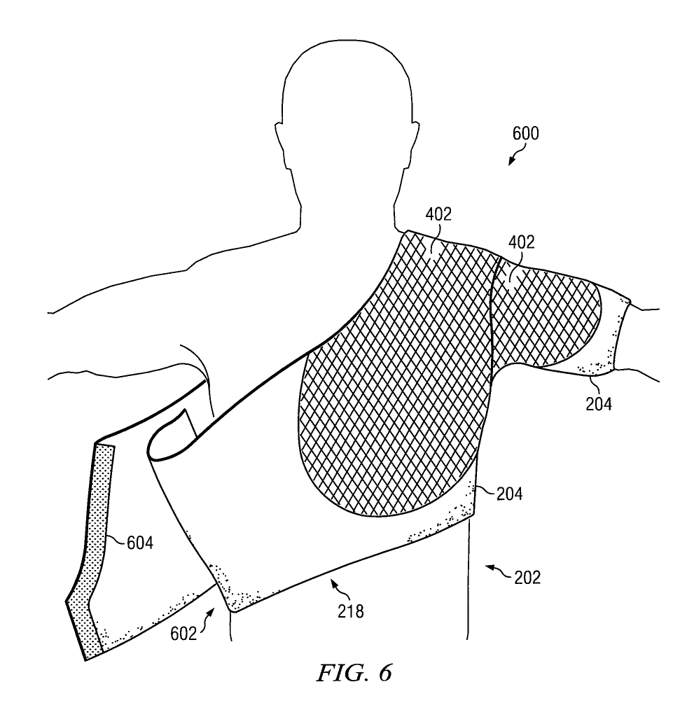

Referring now to FIG. 5, an illustrative embodiment of a panel 410 is depicted in cross-sectional form. The outermost layer 500, which forms outward face 404, comprises a flexible fabric or material that is rugged, yet aesthetic. Layer 500 must be suitable to withstand wear and tear, repetitive cleanings, etc., while protecting the layer(s) beneath it. In the embodiments illustrated herein, layer 500 comprises a polyurethane material with a cotton fabric backing. Immediately under layer 500 is layer 502, which is a rigidity layer. Layer 502 is of made of material of sufficient thickness, density and rigidity to provide the desired compression characteristics detailed herein. In the embodiments illustrated herein, layer 502 comprises one or more foam sub-layers. For example, layer 502 may comprise flexible foam sandwiched between expanded polystyrene and ethylene/vinyl acetate (EVA).

Immediately under layer 502 is layer 504, which is an insulation layer. Layer 504 comprises material that maintains cold within area 408, and retards or prevents the escape of cold through panel 410. Layer 504 may comprise any suitable insulating material—such as foam, foil, synthetic, or fiberfill materials. In the embodiments illustrated herein, layer 504 comprises an aluminum foil-based insulating material. Immediately under layer 504 is layer 506, which is an exposure layer, forming an inner surface of area 408. Layer 506 is directly exposed to either the icing medium or icing bladder, and comprises material suitable to withstand wear and tear, cold temperature, and potential wet conditions, while protecting the layer(s) above it. In the embodiments illustrated herein, layer 506 comprises polyester, “rip-stop” type material.

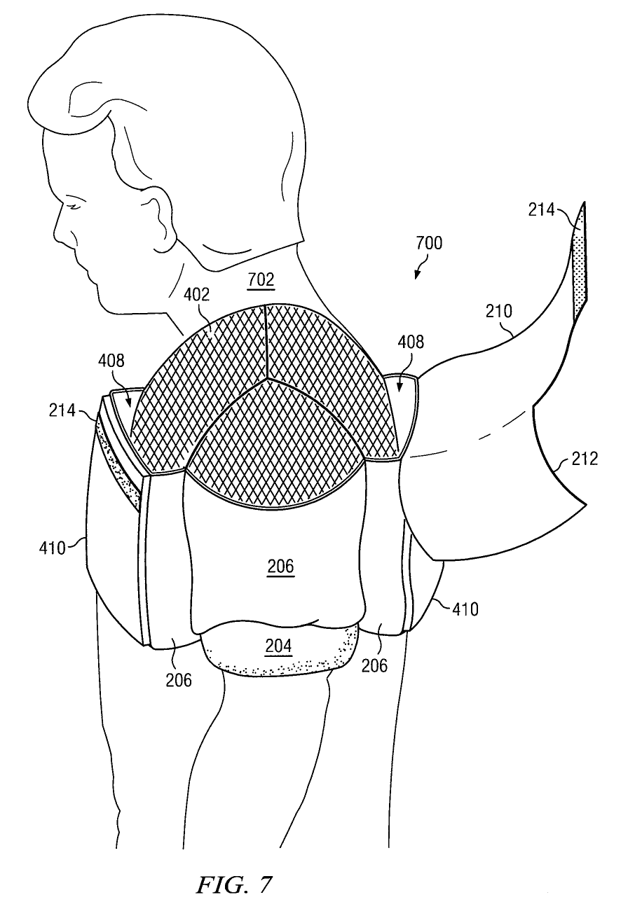

Referring now to FIG. 6, certain aspects of apparatus 200 are described now in greater detail. FIG. 6 depicts an illustrative view 600 of apparatus 200, in which the compression component(s) 208 and outer portions of receptacle(s) 206 are not shown. Positioning component(s) 204 are shown, as are the mesh inward faces 402 of the receptacles, integrated or otherwise attached to component(s) 204. Component(s) 204 comprise open flap portions 602 that form an opening for an individual to put the apparatus on. As depicted, flap portions 602 overlap each other, wrap securely around the torso 202, and are fastened in place via an attachment component 604. In other embodiments, flap portions 602 may abut one another, without overlapping, when closed and secured. As depicted, attachment component 604 comprises a Velcro™ patch that affixes to the exterior of a neoprene opposite flap portion 602 at closure area 218. In other embodiments, attachment component 604 may comprise buckles, snaps, zippers, hooks or any other closure mechanism that provides secure and comfortable closure of the apparatus.

In the embodiment depicted, component(s) 204 are formed of a nylon-covered neoprene material, of sufficient thickness and pliability to secure the orientation/position of receptacle 206, even when the individual is moving around, and to provide support and comfort. Furthermore, faces 402 are formed and positioned to provide icing exposure and contact to an expansive area of torso 202.

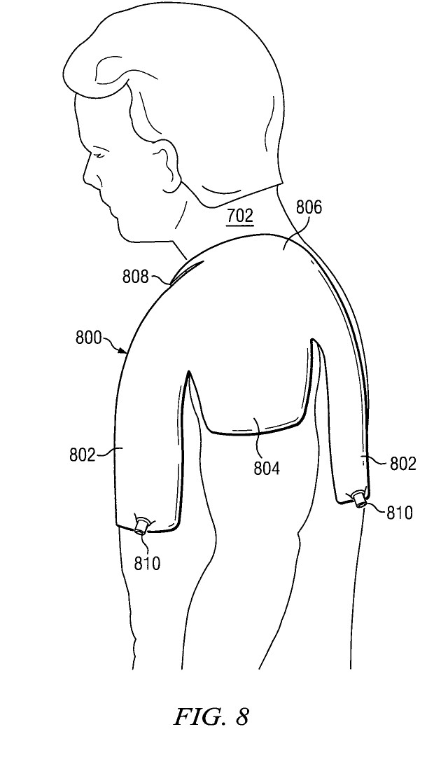

For purposes of illustration and explanation, FIG. 7 presents a perspective view of the apparatus 700 of the present invention in an in-use position upon an individual 702. The inner areas 408 are aligned along the expansive area from the pectoral region, up and over the shoulder region, to the scapular region. Mesh faces 402 are intact along the flesh or undergarment surface of individual 702 that is to be therapeutically treated. Once apparatus 700 is secured in place, an icing medium or icing bladder may be disposed within receptacles 206, after which closures 210 and 212 may be moved into place and secured by closure member 214. In this embodiment, closure member 214 comprises a Velcro™ type closure. Apparatus 700 is provided such that loading, and re-loading, of an icing medium may be accomplished while individual 702 is wearing the apparatus. As such, for convenience and stability, this embodiment is configured in a top-loading fashion. Other embodiments may be provided in alternative arrangements, such as front loading or side loading.

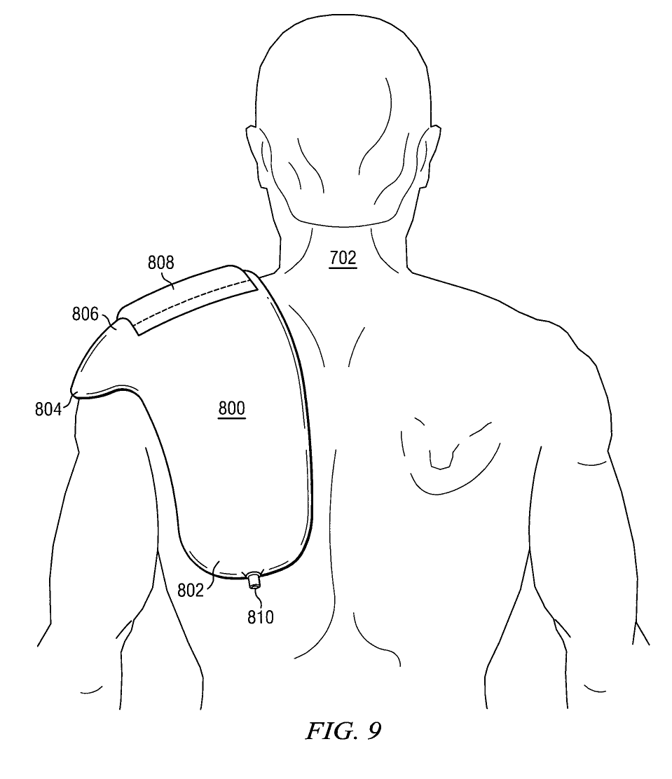

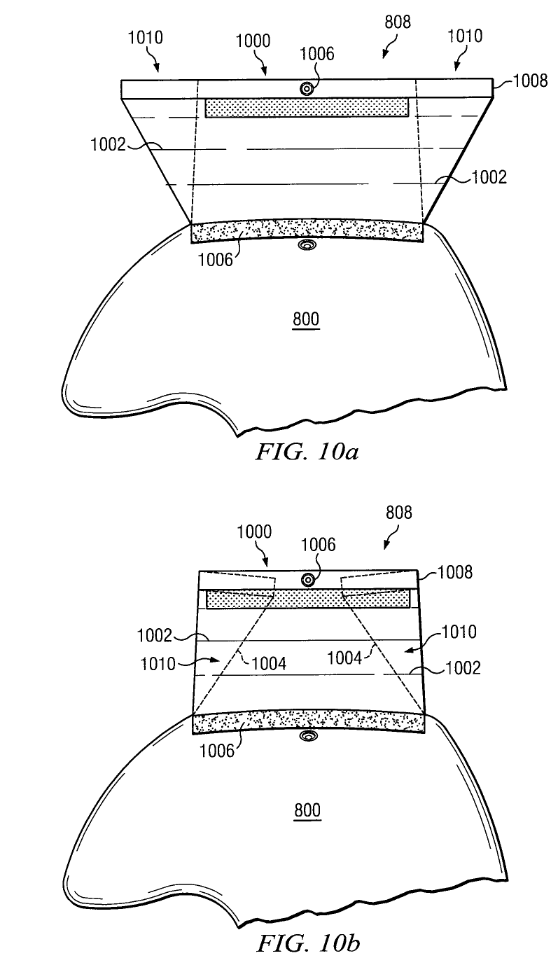

Referring now to FIGS. 8 and 9, one embodiment of a container component 800, a removable icing bladder in this instance, is illustrated in accordance with the present invention. This embodiment of bladder 800 is shaped and formed to operate in conjunction with apparatus 700 (not shown), and is thus illustrated in relation to individual 702. Bladder 800 comprises lateral portions 802 that symmetrically cover the pectoral and scapular areas of individual 702, and a shoulder portion 804 that covers the deltoid area of individual 702. A central upper portion 806 covers the trapezius area of individual 702 once a closeable loading assembly 808 is closed and secured.

In this embodiment, bladder 800 is positioned within apparatus 700 such that lateral portions 802 are evenly positioned with the large receptacles 206, and shoulder portion 804 is positioned within the small receptacle 206. Once bladder 800 is in place within apparatus 700, ice may be loaded into the top of bladder via assembly 808, which is described in greater detail hereinafter. Bladder 800 is formed of material that is sufficiently rugged to withstand repetitive thermal changes and substantial wear and tear. In most embodiments, bladder 800 is formed to be substantially or completely waterproof, so as to avoid inconvenience and discomfort from leaking. Thus, in some embodiments, bladder 800 may be formed from an injection-molded rubber material. In other embodiments, it may be formed from a waterproof textile that is seam-welded together. Still other embodiments may utilize other materials and other configurations.

Bladder 800 may optionally comprise one or more drain components 810 that allow melted icing medium (i.e., ice water) to be selectively drained out of the bladder without removing the bladder 800 or apparatus 700 from the individual 702. Drain components 810 may comprise simple plastic pinch-valves or spigots, or any other suitable components that allow for selective draining of bladder 800. Component 800 may further comprise one or more internal baffles (not shown). These internal baffles may be provided so as to control the expansion, or maintain the relative shape, of component 800 as ice is loaded therein. The baffles may also be provided in such a manner as to reduce the amount of ice necessary within component 800 to achieve optimal therapeutic effects. Component 800 may also optionally comprise other shape-retention components—such as semi-rigid ribs or panels—embedded within component 800.

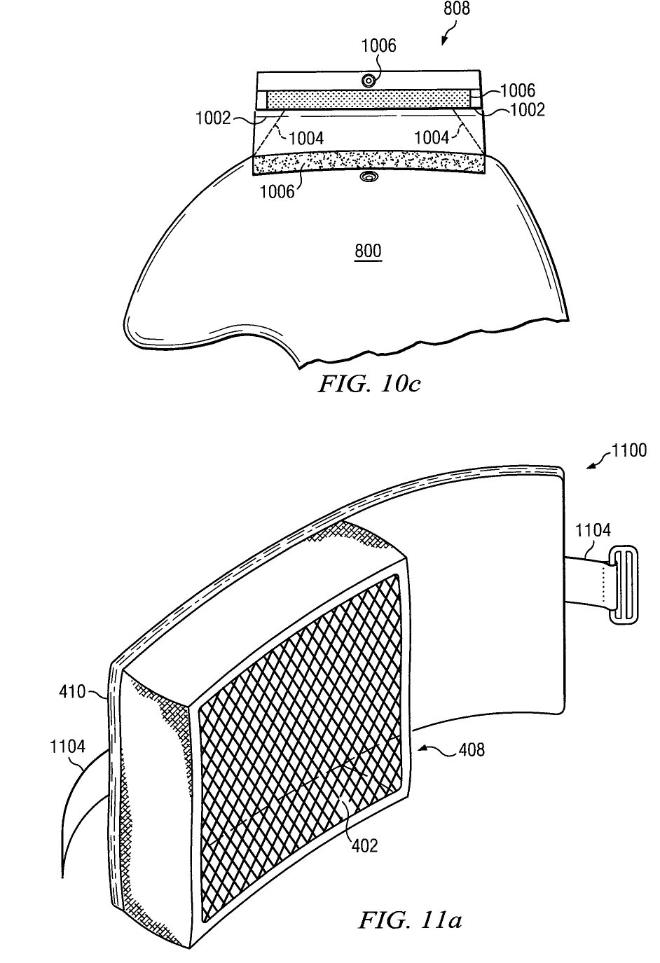

Referring now to FIGS. 10(a)-10(c), one embodiment of a closable loading assembly 808 and several stages of its deployment are illustratively depicted. Assembly 808 is provided such that it may be securely closed in position as previously depicted in FIG. 9. Assembly 808 is formed and configured such that opens outwardly from bladder 800, preferably in a manner that provides a large, chute type, top-loading opening 1000 when fully deployed. In other embodiments, assembly 808 may be provided in a manner that provides a large, chute type opening for other loading orientations, such as front-loading or side-loading. Assembly 808 comprises a number of fan-fold features 1002 that enable a user to close the assembly in upon itself 1004 once filling is complete. Assembly 808 further comprises one or more closure components 1006—such as snaps, Velcro™, zippers, etc.—that provide secure mechanical and thermal closure of bladder 800. Assembly 808 may also comprise a rigid or semi-rigid upper seam 1008 to facilitate stability of the assembly when in its open deployed state.

As depicted in FIG. 10(a), assembly 808 is in an open deployed state. Ice may be loaded, through chute-type opening 1000, into bladder 800. Once filling is concluded, outer portions 1010 are folded in upon themselves 1004 along fold features 1002, as depicted now in FIG. 10(b). The top of assembly 808, from upper seam 1008, is then alternately folded over upon itself along fold features 1002, and one or more closure components 1006 may be engaged. Finally, in FIG. 10(c), the last remaining fold is completed and a closure component 1006 is engaged to keep the assembly in its closed, in-use position.

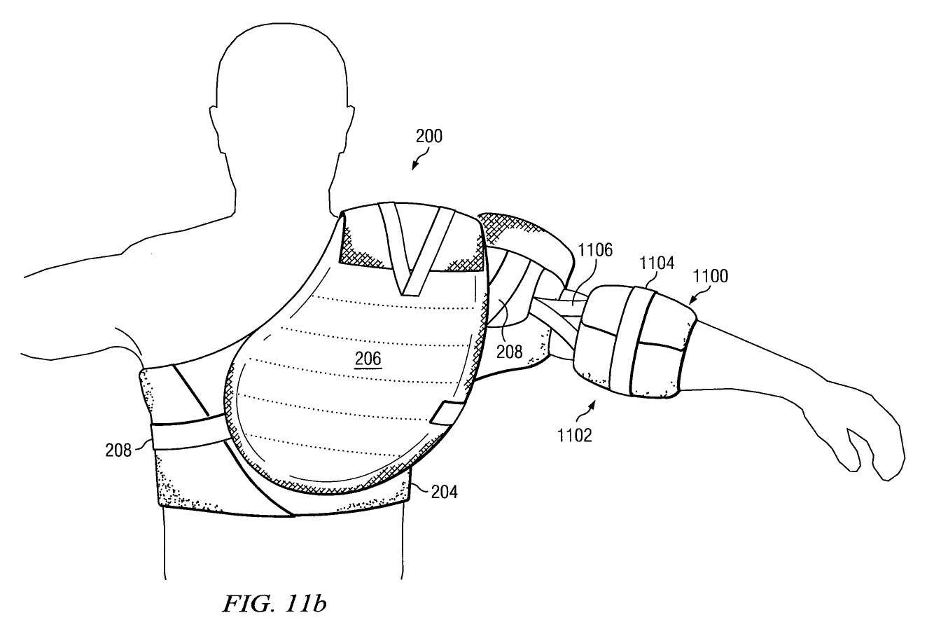

Referring now to FIGS. 11(a) and 11(b), another embodiment of an icing assembly 1100 in accordance with the present invention is illustrated. In this embodiment, assembly 1100 is provided for icing the elbow region 1102 of an individual. Assembly 1100 comprises an exterior panel 410, an inward face 402, and an interior area 408. An icing medium or container component is placed within area 408, and assembly 1100 is wrapped around the elbow area 1102 and closed. Assembly 1100 comprises a closure component 1104 that to secure the assembly in a closed position. In the illustrated embodiment, component 1104 functions both as a closure component and a compression component. In other embodiments, assembly 1100 may have only a closure component, a closure component in conjunction with one or more independent compression components, or one or more compression components without a closure component. Other variations are hereby comprehended.

Apparatus 1100 may be worn independently, or it may be worn in conjunction with another apparatus 200. It may be desirable to provide one or more positioning components 1106 to operationally attach the apparatus together. In other embodiments, apparatus 1100 may be formed as an integral part of apparatus 200.

In all of the embodiments of the present invention, a variety of adaptations and configurations are comprehended and anticipated by this disclosure. For example, any component, assembly or member may be formed integrally with another, or may be formed individually and either removably or permanently attached thereto. Any number of materials or assemblies may be substituted in place of those illustrated, so long as they provide the same form, function or characteristics. Although the embodiments disclosed are illustrated in reference to treatment of a torso area, those same embodiments may be applied with equal effectiveness to treatment of other areas or regions of the body. It is further comprehended that, although the embodiments disclosed are illustrated in reference to a human body, it is within the scope of the present invention that such treatments may be applied to non-human bodies (e.g., race horses).

Thus, while this invention has been described with a reference to illustrative embodiments, this description is not intended to be construed in a limiting sense. As explained above, various modifications and combinations of the illustrative embodiments, as well as other embodiments of the invention, will be apparent to persons skilled in the art upon reference to the description. It is, therefore, intended that the appended claims encompass any such modifications or embodiments.

What is claimed is:

1. A system for therapeutically cooling a desired area on an individual's body, comprising:
a closable therapeutic medium receptacle, having insulating and compression features;
a positioning component connected to the therapeutic medium receptacle, and adapted to maintain the therapeutic medium receptacle in a desired orientation with respect to the desired area; and
a compression component connected to the therapeutic medium receptacle, and adapted to facilitate compression of the therapeutic medium receptacle against the desired area.

2. The system of claim 1 wherein the closable therapeutic medium receptacle is formed such that a therapeutic medium may be disposed directly within the closable therapeutic medium receptacle.

3. The system of claim 1 further comprising a container component, formed to contain a therapeutic medium, and disposed with the closable therapeutic medium receptacle.

4. The system of claim 1 wherein a therapeutic medium disposed within the closable therapeutic medium receptacle comprises ice.

5. The system of claim 3 wherein the container component is formed to be removable from the closable therapeutic medium receptacle.

6. The system of claim 3 wherein the container component is a removable bladder.

7. The system of claim 3 wherein the container component comprises a closable loading assembly.

8. The system of claim 7 wherein the closable loading assembly is formed to close by folding in upon itself.

9. The system of claim 1 wherein the closable therapeutic medium receptacle comprises a panel having an insulating layer and a compression layer.

10. The system of claim 9 wherein compression layer comprises a foam-type material.

11. The system of claim 9 wherein compression layer comprises a plastic material.

12. A method of therapeutically cooling a desired area on an individual's body, comprising the steps of:
providing a therapeutic medium receptacle, having insulating and compression features;
providing a positioning component connected to the therapeutic medium receptacle, and adapted to maintain the therapeutic medium receptacle in a desired orientation with respect to the desired area;
providing a compression component connected to the therapeutic medium receptacle, and adapted to facilitate compression of the therapeutic medium receptacle against the desired area;
loading a therapeutic medium into the therapeutic medium receptacle; and
securing the therapeutic medium receptacle, the positioning component, and the compression component in place on the individual's body.

13. The method of claim 12 wherein a therapeutic medium is disposed directly within the therapeutic medium receptacle.

14. The method of claim 12 further comprising the step of providing a container component, formed to contain a therapeutic medium, and to be disposed with the therapeutic medium receptacle.

15. The method of claim 12 wherein a therapeutic medium disposed within the therapeutic medium receptacle comprises ice.

16. The method of claim 14 wherein the container component is removable from the therapeutic medium receptacle.

17. The method of claim 12 wherein the step of providing a therapeutic medium receptacle further comprises providing a panel having an insulating layer and a compression layer.

18. The method of claim 12 further comprising the steps of:
providing a second therapeutic medium receptacle;
loading a therapeutic medium into the therapeutic medium receptacle; and
securing the therapeutic medium receptacle in place on the individual's body.

19. The method of claim 18 wherein the step of providing a second therapeutic medium receptacle further comprises providing the second therapeutic medium receptacle medium attached to the therapeutic medium receptacle.

20. An apparatus for therapeutically icing an extensive area of an individual body, comprising:
a therapeutic medium receptacle, having an inward face formed of a thermally penetrable material, having an outward face, and having an insulating and compression panel disposed along the outward face, adapted to receive and contain a therapeutic icing medium;
a positioning component attached to the therapeutic medium receptacle, having a closure component to secure the apparatus in place along the individual body, and adapted to maintain the therapeutic medium receptacle in a desired orientation with respect to the extensive area; and
a plurality of compression components connected to the therapeutic medium receptacle and positioned about the individual body to facilitate compression of the compression panel against the extensive area.
